(19) United States
(12) Patent Application Publication
(10) Pub. No.: US 2025/0331530 A1
(43) Pub. Date: Oct. 30, 2025

(54) DEAMIDATED WHEY PROTEIN POWDER WITH ENHANCED THERMAL STABILITY

(71) Applicant: Purdue Research Foundation, West Lafayette, IN (US)

(72) Inventor: Da Chen, West Lafayette, IN (US)

(73) Assignee: Purdue Research Foundation, West Lafayette, IN (US)

(21) Appl. No.: 19/186,916

(22) Filed: Apr. 23, 2025

Related U.S. Application Data
(60) Provisional application No. 63/638,649, filed on Apr. 25, 2024.

(51) Int. Cl. A23C 21/02; A23L 2/56; A23L 2/58; A23L 2/60; A23L 2/66; C12N 9/80

(52) U.S. Cl. CPC A23C 21/02; A23L 2/56; A23L 2/58; A23L 2/60; A23L 2/66; C12N 9/80; A23C 2220/10; C12Y 305/01002

(57) ABSTRACT

A method for preparing deamidated sweet whey protein powder via enzymatic deamidation; a whey protein beverage exhibiting high clarity and is essentially turbidity-free, and its method of preparation using the deamidated sweet whey protein powder.

### CROSS-REFERENCE TO RELATED APPLICATION

This application claims priority to U.S. provisional patent application No. 63/638,649, which was filed Apr. 25, 2024, and which is hereby incorporated by reference in its entirety.

### TECHNICAL FIELD

The present disclosure relates to a deamidated whey protein powder with enhanced thermal stability, its method of preparation via enzymatic deamidation, and its use for preparing beverages with no turbidity.

### BACKGROUND

This section introduces aspects that may help facilitate a better understanding of the disclosure. Accordingly, these statements are to be read in this light and are not to be construed as admissions about what is or is not prior art.

Nutritional beverages are experiencing rapid growth (˜6.5% annually) in the market. Whey protein-based beverages have become highly valued for their protein quality and health benefits, such as increased satiety, protection against muscle-protein loss, enhanced muscle-protein synthesis, and improved glycemic control (International Dairy Journal, 2018, 85, 144-152; Journal of Food Science, 2010, 75 (1), C21-C27). Pasteurization is commonly used to prevent microbial growth and extend the shelf life of whey beverages with a typical heating condition of 80-90° C. for 45 seconds to 2 minutes or 120-150° C. for 2-4 seconds (Advanced Dairy Chemistry, 2026, Volume 1B, Proteins: Applied Aspects, 247-286). Pasteurization used during low-acid whey protein beverage development commonly causes turbidity and sedimentation of the beverages due to protein denaturation and aggregation, leading to deteriorated quality attributes (International Journal of Biological Macromolecules, 1991, 13 (3), 165-173).

Heating above 70° C. enables the unfolding of whey proteins and exposes buried hydrophobic regions and reactive thiol groups. Hydrophobic interactions and thiol/disulfide exchange reactions thus occur and promote aggregation of whey proteins. When the aggregates grow large enough, they start to scatter light and settle out of water to form sedimentation. Increasing the surface charge of whey proteins by pH-shifting or salt addition alleviates heat-induced aggregation due to increased electrostatic repulsion, but this could increase the astringency or salty taste of whey beverage (Carter et al., Journal of Dairy Science, 2020,103 (7), 5793-5804). Other approaches, such as complexation/conjugation with polysaccharides (Gentes et al., Journal of Agricultural and Food Chemistry, 2010, 58 (11), 7051-7058; Liu and Zhong, Food Hydrocolloids, 2015, 44, 453-460), adding chelating or thickening agents (Rasouli et al., International Journal of Dairy Technology, 2020, 73 (1), 46-56), incorporating co-proteins (casein) (Singh et al., Food Research International, 2019, 116, 103-113), enzymatic hydrolysis (Kleekayai et al., Food Hydrocolloids, 2023, 109351), and microparticulation (Wijayanti et al., Food Science and Food Safety, 2014, 13 (6), 1235-1251) have also been used. They either require strict formulation control or additional labeling in the package, consume extensive energy, or negatively affect the mouthful feeling of whey protein beverages. Developing a whey protein ingredient with high thermal stability is essential to produce a beverage with desirable clarity.

It is an object of a present disclosure to provide a deamidated whey protein powder with enhanced thermal stability and a green, simple, and scalable method for its preparation and its use for preparing beverages with no turbidity. This and the other objects and advantages, as well as inventive features, will be apparent from the detailed description.

### SUMMARY

Provided is a method for producing a deamidated sweet whey protein powder (DSWPP), which method comprises:
(i) heating an aqueous solution of a concentrated sweet whey and an enzyme protein glutaminase (e.g., PG-500) at about 40° C. to about 60° C. to obtain a solution of deamidated sweet whey; and
(ii) either spray-drying or freeze-drying the solution of deamidated sweet whey,
whereupon the deamidated sweet whey protein powder (DSWPP) is produced.

In some embodiments, the concentrated sweet whey comprises a protein content of about 5% w/v to about 15% w/v. The amount of the protein glutaminase used can be from about 5 U/g protein to about 50 U/g protein. The DSWPP can comprise about 70% to about 90% of proteins. The solution of deamidated sweet whey of step (i) can be heated for about 1 hour.

Provided is a whey protein beverage comprising about 3% by weight to about 10% by weight of a whey protein, wherein the whey protein is from a deamidated sweet whey protein powder (DSWPP), a sweetener and having a turbidity value of less than about 0.1 a.u. The whey protein beverage can further comprise one or more additives. The sweetener can be selected from a natural sweetener, such as a sugar and an artificial sweetener. One or more additives can be selected from a nutrient, a herbal supplement, a flavoring agent, a coloring agent, and a combination of two or more thereof. The whey protein beverage is clear and has a turbidity value of less than about 0.1 a.u. for a time period from about a week to about 1.5 months during storage with or without refrigeration. The DSWPP produced in accordance with the above-described method is used to prepare the whey protein beverage.

The whey protein beverage can be a protein shake or a protein cream. In some embodiments, the protein shake and the protein cream comprise the whey protein beverage described herein. A method of producing a whey protein beverage is provided. The method comprises:
a. preparing an aqueous solution of a deamidated sweet whey protein powder (DSWPP) to achieve a protein content of about 3% w/v to about 10% w/v in the solution;
b. combining the aqueous solution of DSWPP with a sweetener to produce a whey protein beverage; and
c. subjecting the whey protein beverage to an ultra-high temperature (UHT) processing.

The method further comprises an addition of one or more additives. The whey protein beverage can be produced at a neutral pH and has a turbidity value of less than about 0.1 a.u. The DSWPP used to prepare the whey protein beverage is produced in accordance with the above-described method.

Also provided is a method for producing a deamidated whey protein powder (DWPP), which method comprises:
(i) heating an aqueous solution of a whey protein comprising a protein content of about 5% w/v to about 15% w/v and an enzyme protein glutaminase (e.g., PG-500) at about 40° C. to about 60° C. to obtain a solution of deamidated whey protein; and
(ii) either spray-drying or freeze-drying the solution of deamidated whey protein,

### BRIEF DESCRIPTION OF DRAWING

The present disclosure will be more readily understood from the detailed description of embodiments presented below, considered in conjunction with the attached drawings of which:

FIG. 1A shows a graph of the turbidity of whey protein solution (5% w/v) containing various amounts of glutaminase PG-500 heated at 85° C. for 10-30 minutes.

FIG. 1B are images of the turbidity of whey protein solution (5% w/v) containing various amounts of PG-500 heated at 85° C. for 20 minutes.

FIG. 2 shows a graph (top) and the images (bottom) of the storage stability of heated whey protein solution (5% w/v, 85° C. for 20 minutes) at 5° C. Control (ctrl) is the whey protein solution without PG-500. 50 U/g protein is a whey protein treated with PG-500 at the concentration of 50 U/g protein. Day 1, is the day whey protein solution was heated at 85° C. for 20 min.

FIG. 3 shows the heat stability of freeze-dried whey protein powder pre-treated with various concentrations of PG-500. The samples were reconstituted in water to a 5% protein concentration, followed by heating at 85° C. for 20 min.

FIG. 4A shows the degree of deamidation of whey proteins treated with various amounts of PG-500. Different letters represent significant differences among the data.

FIG. 4B shows the degree of hydrolysis of whey proteins treated with various amounts of PG-500.

FIG. 4C shows the reducing SDS-PAGE profile of whey proteins treated with various amounts of PG-500.

FIG. 5A shows the synchrotron ultra-small and small-angle X-ray scattering of whey protein solution (5%) containing various amounts of PG-500 after heating at 85° C. for 20 minutes.

FIG. 5B shows the volume size distribution of heated whey protein solutions derived from the fitting of X-ray scattering.

FIG. 5C is a schematic of deamidated whey protein showing the reduced size of aggregates after heating.

FIG. 6A shows the solubility of heated whey protein solution (5% w/v) (85° C., 20 minutes) containing various amounts of PG-500. Different letters denote significant differences among the data (P<0.05).

FIG. 6B shows the zeta potential of heated whey protein solution (5% w/v) containing various amounts of PG-500.

FIG. 6C shows surface hydrophobicity of heated whey protein solution (5% w/v) containing various amounts of PG-500.

FIG. 6D shows intrinsic fluorescence of whey protein solution (5% w/v) containing various amounts of PG-500 before (no heat) and after heating at 85° C. for 20 minutes. Different letters denote significant differences among the data (P<0.05).

FIG. 7 shows the turbidity and appearance of whey protein solution prepared from spray-dried whey protein powder with and without heating (75° C. for 30 minutes). The whey proteins were deamidated with different amounts of PG-500 prior to spray drying. The letter C in legends indicates that the whey protein solution was centrifuged prior to heating.

FIG. 8 shows the particle size distribution of re-suspended whey protein solution prepared from spray-dried whey protein powder after heating at 75° C. for 30 minutes. The left graph represents 5% w/v whey protein solution without centrifuge prior to heating, and the right graph represents 5% w/v whey protein solution subjected to centrifugation prior to heating.

FIG. 9 shows transmission electron microscopy images of spray-dried whey protein powder solution (0.1 mg/mL) containing various amounts of PG-500 after heating (75° C., 30 minutes). The whey proteins were deamidated with different amounts of PG-500 prior to spray drying. No centrifugation was applied between.

FIG. 10 shows the zeta potential of spray-dried whey protein powder solution containing various amounts of PG-500 before (no heat) and after heating (75° C., 30 minutes). Different letters like a and f in the graph denote significant differences among the data (P<0.05).

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the claimed invention is thereby intended.

The present disclosure is predicated, at least in part, on the discovery that the deamidation process can be applied to food proteins to improve their solubility and interfacial properties by converting glutamine and/or asparagine to glutamic acid and aspartic acid (Chen et al., Comprehensive Reviews in Food Science and Food Safety, 2021, 20 (4), 3788-3817). Such conversion could be fulfilled by using chemicals (acid, alkaline, ion-exchange resin) or enzymes (e.g., glutaminase). The deamidation process can enhance umami of wheat proteins (Liu et al., Journal of the Science of Food and Agriculture, 2017, 97 (10), 3181-3188) and can reduce the lumpiness and grittiness of rice proteins (Hu et al., International Journal of Food Science and Technology, 2019, 54 (7), 2458-2467). However, whey proteins possess distinct molecular structures compared to these plant proteins.

Provided is a method of enzymatic deamidation of a whey protein that is simple, scalable, and cost-effective. The method can enhance the thermal stability of the whey protein. The enzyme-protein ratio can be important to achieve stability improvement of protein solution. The deamidated whey protein powder (DWPP) can be prepared by enzymatic deamidation of whey proteins using protein glutaminase (e.g. glutaminase PG-500). The whey protein used can be any suitable type of whey protein. The whey protein comprises a protein content of about 5% w/v to about 15% w/v. The whey protein can be from milk serum, sweet whey, or acid whey.

Provided is a method for producing a deamidated whey protein powder (DWPP), which method comprises:
(i) heating an aqueous solution of a whey protein comprising a protein content of about 5% w/v to about 15% w/v and an enzyme protein glutaminase (e.g., PG-500) at about 40° C. to about 60° C. to obtain a solution of deamidated whey protein; and
(ii) either spray-drying or freeze-drying the solution of deamidated whey protein,
whereupon the deamidated whey protein powder (DWPP) is produced.

The whey protein can comprise a protein content of about 5% w/v to about 15% w/v, such as about 5% w/v to 15% w/v, 5% w/v to about 15% w/v, or 5% w/v to 15% w/v. In some embodiments, the whey protein is heated with enzyme glutaminase (e.g., PG-500) at a temperature of about 40° C. to about 60° C., such as about 40° C. to 60° C., 40° C. to about 60° C., or 40° C. to 60° C. In some embodiments, the whey protein solution is heated at about 55° C. (e.g., 55° C.) for about 1 hour (such as 1 hour). The solution of deamidated whey protein can be subjected to spray drying or freeze drying to form deamidated whey protein powder. Provided is a deamidated whey protein powder (DWPP) comprising a protein content of about 70% to about 90%, such as about 70% to 90%, 70% to about 90%, or 70% to 90%. In some embodiments, the DWPP has high thermal stability. The DWPP can be used directly to prepare food products such as beverages.

Any suitable whey can be used. In some embodiments, the whey can be a sweet whey. Sweet whey is formed during cheese production. It is a byproduct of the manufacture of rennet types of hard cheese, e.g., cheddar cheese or Swiss cheese. Sweet whey by mass contains about 93% water, about 0.8% protein, about 5.1% carbohydrates (e.g., lactose), about 0.4% fat, and has a pH greater than or equal to 5.6. It also contains some minerals. Sweet whey has a high lactose content compared to its protein content. The sweet whey can be concentrated to increase the protein content.

Provided is a method for preparing a deamidated sweet whey protein powder (DSWPP), which method comprises:
(i) heating an aqueous solution of a concentrated sweet whey and an enzyme protein glutaminase (e.g., PG-500) at about 40° C. to about 60° C. to obtain a solution of deamidated sweet whey; and
(ii) either spray-drying or freeze-drying the solution of deamidated sweet whey,
whereupon the deamidated sweet whey protein powder (DSWPP) is produced.

The concentrated sweet whey can comprise a protein content of about 5% w/v to about 15% w/v, such as about 5% w/v to 15% w/v, 5% w/v to about 15% w/v, or 5% w/v to 15% w/v. In some embodiments, the concentrated sweet whey comprises a protein content of 5% w/v. In some embodiments, the concentrated sweet whey comprises a protein content of 8% w/v. In some embodiments, the concentrated sweet whey comprises a protein content of 10% w/v. In some embodiments, the concentrated sweet whey comprises a protein content of 12% w/v. In some embodiments, the concentrated sweet whey comprises a protein content of 15% w/v. In some embodiments, the concentrated sweet whey is heated with enzyme glutaminase PG-500 at a temperature of about 40° C. to about 60° C., such as about 40° C. to 60° C., 40° C. to about 60° C., or 40° C. to 60° C. In some embodiments, the concentrated sweet whey solution is heated with enzyme glutaminase PG-500 at about 55° C. (e.g., 55° C.) for about 1 hour (such as 1 hour). The solution of deamidated sweet whey can be subjected to spray drying or freeze drying to form deamidated sweet whey protein powder.

The enzyme glutaminase PG-500 used for the deamidation process can be derived from C. proteolyticum and verified to be safe for oral ingestion. The enzyme catalyzes the deamidation of glutamine but not asparagine and can be on whey proteins, casein, α-lactobulins, milk proteins, soy proteins, pea proteins, oat proteins, and zein. The PG-500 can convert glutamine to glutamic acid and increase the number of carboxylic groups in whey proteins. This can potentially increase the surface charge and alter protein unfolding in response to heat. Whey proteins possess distinct molecular structure compared to other plant proteins. After enzymatic deamidation, the deamidated whey protein can repulse each other more and form less covalent and non-covalent bonds under heat due to increased surface charge, which can lead to the formation of smaller aggregates exhibiting improved water solubility and stability suitable to develop high-clarity protein beverages. The whey protein beverage commonly develops turbidity and sedimentation due to protein denaturation and aggregation. However, the enzymatic deamidation process can increase the thermal stability of whey protein, as described above. Thus, whey protein beverages produced with deamidated whey protein powder can have high clarity with substantially no turbidity. The concentration of glutaminase can control the degree of deamidation.

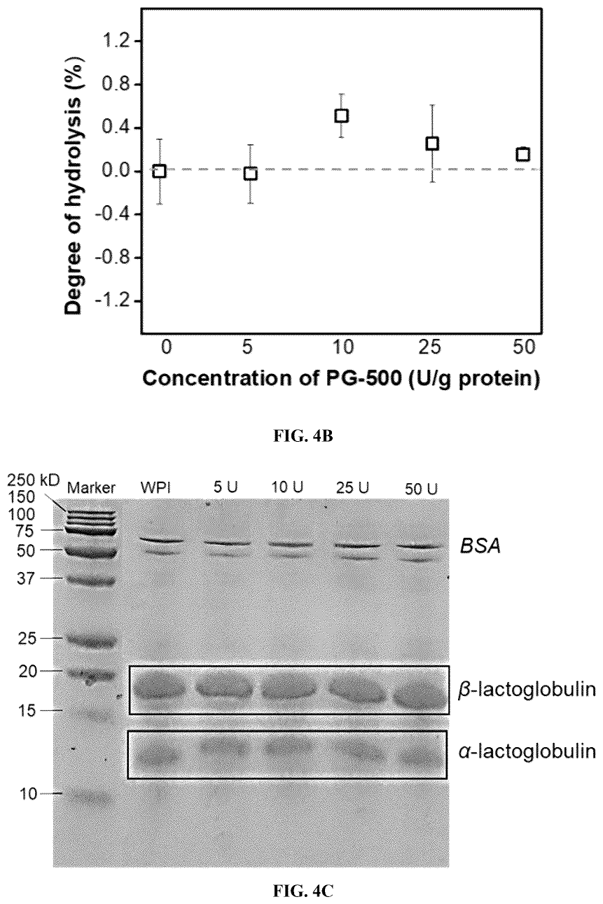

The protein glutaminase PG-500 can be used at a concentration of about 5 U/g protein to about 50 U/g protein (e.g., about 5 U/g protein to 50 U/g protein, 5 U/g protein to about 50 U/g protein, 5 U/g protein to 50 U/g protein). In some embodiments, the concentration of PG-500 is 5 U/g protein. In some embodiments, the concentration of PG-500 is 10 U/g protein. In some embodiments, the concentration of PG-500 is 15 U/g protein. In some embodiments, the concentration of PG-500 is 20 U/g protein. In some embodiments, the concentration of PG-500 is 25 U/g protein. In some embodiments, the concentration of PG-500 is 30 U/g protein. In some embodiments, the concentration of PG-500 is 35 U/g protein. In some embodiments, the concentration of PG-500 is 40 U/g protein. In some embodiments, the concentration of PG-500 is 45 U/g protein. In some embodiments, the concentration of PG-500 is 50 U/g protein. The dose-dependent effect of PG-500 on the improvement of the clarity of the whey protein solution was mainly due to a distinct degree of deamidation (FIG. 4a). In some embodiments, the degree of deamidation can be from about 5% to about 15%, such as about 5% to 15%, 5% to about 15%, or 5% to 15%. No hydrolysis of whey protein was observed, which was evidenced by a near-zero DH value and the intact beta-lactoglobulin and alpha-lactalbumin bands in the SDS-PAGE (FIGS. 4b-4c).

Provided is a deamidated sweet whey protein powder (DSWPP) comprising a protein content of about 70% to about 90%, such as about 70% to 90%, 70% to about 90%, or 70% to 90%. In some embodiments, the DSWPP has high thermal stability. The DSWPP can be used directly to prepare food products such as beverages. The powder can reduce flavor binding problems encountered in high protein-containing foods and beverages.

Further provided is a whey protein beverage comprising a deamidated sweet whey protein (DSWP) at an amount effective in preventing the formation of turbidity. The whey protein beverage comprises about 3% by weight to about 10% by weight of a DSWP, a sweetener wherein the whey protein is from DSWPP, which provides the beverage high clarity. The beverage can have a turbidity value of less than 0.1 a.u. The DSWPP can be prepared using the above-described method. Also provided is a whey protein beverage comprising a deamidated whey protein (DWP) at an amount effective in preventing the formation of turbidity. The whey protein beverage comprises about 3% by weight to about 10% by weight of a DWP, a sweetener wherein the whey protein is from DWPP, which provides the beverage high clarity. The beverage can have a turbidity value of less than 0.1 a.u. The DWPP can be prepared using the above-described method.

The sweetener can be a natural sweetener or an artificial sweetener. In some embodiments, the natural sweetener is a sugar. The whey protein beverage can comprise about 20% (e.g., 20%) by weight sugar. Any suitable artificial sweetener can be used. Artificial sweeteners can be selected from aspartame, sucralose, acesulfame K, saccharin, purified stevia leaf extracts, and xylitol. The whey protein beverage can further comprise one or more additional additives. Examples of one or more additives include but are not limited to, a nutrient, a herbal supplement, a flavoring agent, a coloring agent, and a combination of two or more thereof. In some embodiments, whey beverages can be produced without the addition of stabilizers or pH modifications. The beverages can be prepared at about neutral pH (e.g., at pH 7). The DSWP can be added in the powder form. The amount of DSWP can be added from about 3 g to about 12 g, such as about 3 g to 12 g, 3 g to about 12 g, or 3 g to 12 g to achieve a protein content of about 3% by weight to about 10% by weight.

In some embodiments, the whey protein beverage is clear, exhibiting substantially no turbidity. The beverage can be stable with substantially no turbidity for a time period from about a week to about 1-3 months during storage with or without refrigeration. The time period can be from about a week to about 1.5 months (such as a week from 1.5 months) with or without refrigeration. The time period can be from about a week to about 2 months (such as a week from 2 months) with or without refrigeration. The time period can be from about a week to about 2.5 months (such as a week from 2.5 months) with or without refrigeration. In some embodiments, the time period is from a week to 1.5 months with or without refrigeration. The beverage can be stored at a temperature from about 5° C. to about 25° C. such as about 5° C. to 25° C., 5° C. to about 25° C., or 5° C. to 25° C. In some embodiments, the whey protein beverage exhibits a turbidity of less than 0.1 a.u. Examples of whey protein beverages include but are not limited to, protein shakes and protein creams. Provided is a protein shake comprising the whey protein beverage described herein. Provided is a protein cream comprising the whey protein beverage described herein.

Further provided is a method of producing a whey protein beverage, which method comprises:
a. preparing an aqueous solution of a deamidated sweet whey protein powder (DSWPP) to achieve a protein content of about 3% w/v to about 10% w/v in the solution;
b. combining the aqueous solution of DSWPP with a sweetener to produce a whey protein beverage; and
c. subjecting the whey protein beverage to an ultra-high temperature (UHT) processing.

The whey protein beverage can be produced using DWPP, e.g., DSWPP, which is prepared in accordance with the above-described method. The method further comprises adding one or more additives. One or more additives can be a nutrient, a herbal supplement, a flavoring agent, a coloring agent and a combination of two or more thereof. The whey protein beverage can be formulated at a neutral pH. The whey protein beverage can have a turbidity of less than 0.1 a.u. The whey protein beverage exhibits turbidity of less than 0.1 a.u. for a period of from about a week to about 1-3 months during storage with or without refrigeration. The time period can be from about a week to about 1.5 months (such as a week from 1.5 months) with or without refrigeration. The time period can be from about a week to about 2 months (such as a week from 2 months) with or without refrigeration. The time period can be from about a week to about 2.5 months (such as a week from 2.5 months) with or without refrigeration. In some embodiments, the time period is from a week to 1.5 months with or without refrigeration. The DSWPP can be prepared in accordance with the above-described method. The whey protein beverage can be subjected to heat sterilization by UHT processing.

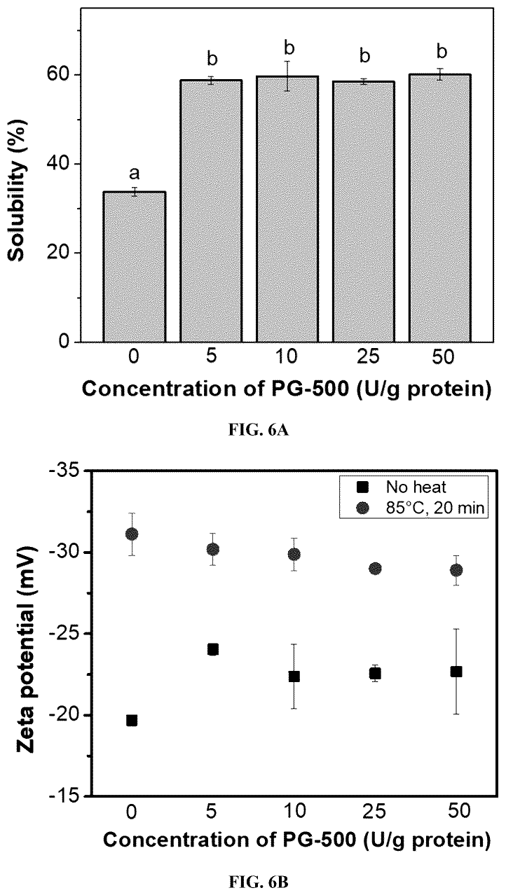

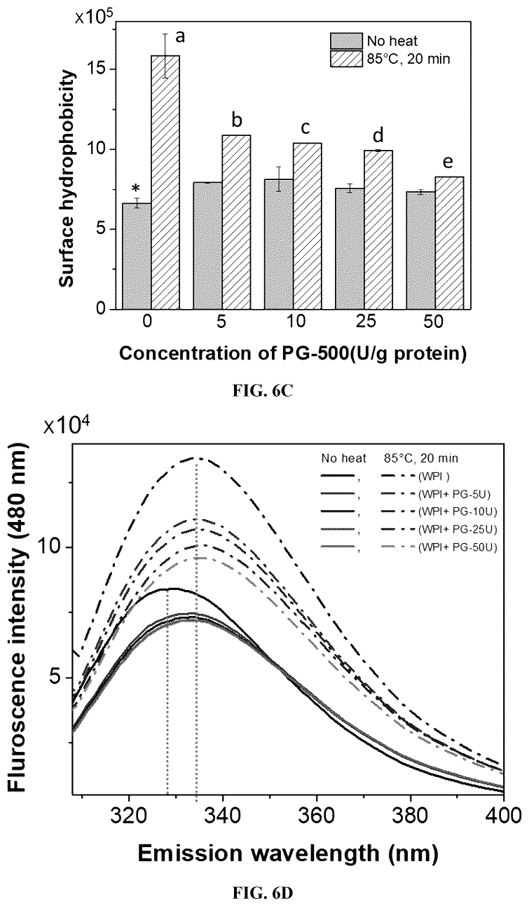

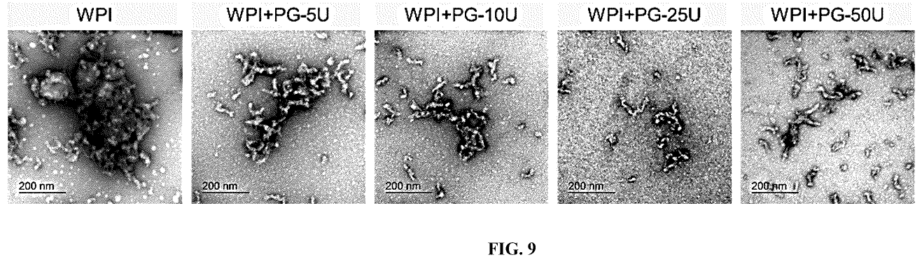

It was observed that for the concentration of 25 U/g protein of PG-500, the turbidity value of the whey protein solution (5% w/v) was 0.13 a.u., and for the concentration of 50 U/g protein of PG-500, the turbidity value of the whey protein solution was 0.18 a.u. The size of the protein aggregates was measured. The whey protein solution, formed using whey protein without PG-500 treatment (PG-500 free), produced aggregates of size ranging between 2.5 nm and 35 nm with a median value of about 8 nm, while the whey protein solution, formed using the whey protein with PG-500 treatment produced smaller aggregates of size ranging between about 2.0 nm and about 10 nm with a median value of about 4 nm. This indicates that deamidation using PG-500 can enhance the heat stability of whey protein and reduce the turbidity of its solution by a reduction in protein aggregation. The solubility of whey protein aggregates was also measured. The solubility was increased from about 32% in PG-500 free whey protein solution to about 60% in those deamidated by PG-500 (FIG. 6a). The increased solubility can be due to the decreased size of the aggregates and/or increased surface charge. Deamidation also improved the light transmittance and clarity of WP particles and evenly distributed small protein aggregates, which indicates the improved thermal stability and improved clarity of the whey protein solution (FIG. 9).

Thus, PG-500 can reduce the heat-induced turbidity of whey protein solution at about 5% protein concentration. Glutaminase (PG-500) induced deamidation can increase the electrostatic repulsion among whey proteins and delay their denaturation, leading to the formation of smaller aggregates with improved water solubility and stability, resulting in decreased turbidity.

### EXPERIMENTAL

The following examples serve to illustrate the present disclosure. The examples are not intended to limit the scope of the claimed invention in any way.

### Examples

Deamidated Whey Protein Powder from Sweet Whey

Sweet whey with solid content (15-20%) was deamidated with PG-500 (25 U/g protein) at 55° C. for 1 hour, followed by drying using a Buchi B290 spray dryer. The freshly spray-dried powder was collected from a collection vessel at the bottom of a cyclone and kept in a sealed bag at 4° C. prior to further analysis). Control samples were without adding PG-500.

### Deamidated Whey Protein

1] Whey protein (WP) solution (5%, w/v) was mixed with various amounts of glutaminase PG-500 and reacted at 55° C. for 1 hour prior to heating at 85° C. for 10-30 minutes.
2] WP was added to 50 mM sodium phosphate buffer (pH7) and stirred (˜300 rpm) for 2 hours to prepare a 5% (w/v) solution. It was centrifuged (10,000×g, 15 min), filtered (0.45 μm filter), incubated at 55° C. for 5 minutes followed by addition of PG-500 in a proportion of 0, 5, 10, 25 and 50 U/g protein, and reacted at 55° C. for 1 hour to achieve various degree of deamidation.

### Degree of Deamidation

The degree of deamidation was measured according to Fu et al., Food Chemistry, 2022, 385, 132512 and Miwa et al. Journal of Agricultural and Food Chemistry, 2013, 61 (9), 2205-2212 with modifications, which are incorporated herein by reference for their teaching regarding the same. After deamidation, the WPI solution was transferred to ice immediately, centrifuged at 4° C. (10,000×g, 10 min), filtered, and diluted 50 times with cold water. The diluents were mixed with ammonia assay reagent and reacted in the dark for 15 min prior to measuring the fluorescence intensity at 360 nm excitation wavelength and 450 nm emission wavelength. For the complete deamidation, WP solution was mixed with the same volume of 4 N HCl, sealed, and heated at 100° C. for 4 hours. The solution was neutralized with 2 M NaOH prior to measuring the ammonia content using the ammonia assay reagent. The degree of deamidation was measured as the ratio of ammonia generated in the PG-500 deamidated sample to those of completely deamidated WP.

### Turbidity Measurement

WP solution with various degrees of deamidation was heated at 85° C. for 10-30 minutes followed by cooling down to 20° C. in a water bath. The solutions were transferred to cuvettes with a path length of 1 cm. The transmittance (%) was measured using a UV-vis spectrophotometer (GENESYS 150,Thermo Scientific, USA) at a 600 nm wavelength. The turbidity (%) was calculated as Turbidity=(100-transmittance)/100 (Chen et al., Biomacromolecules, 2021, 22 (2), 1001-1014).

### Degree of Hydrolysis (DH)

DH was determined using the o-phthaldialdehyde (OPA) method as described by Chen et al. Journal of Functional Foods, 2013, 5 (2), 689-697) with slight modification. Heated WP solution (0.2 mL) (85° C., 20 min) diluted with water and 0.2 mL diluent was added to 1.5 mL OPA reagent and reacted for 2 min at room temperature before recording the absorbance at 340 nm in a spectrophotometer. The absorption of the total amount of amide groups in the hydrolysates was also measured using the same protocol after completely hydrolyzing with 6 N HCl for 24 hours at 120° C. DH calculated using the following formula:

$$D H = {A}_{sample} / ( {A}_{Complete hydrolysis} \times dilution factor ) \times 100 \%$$

### Sodium Dodecyl Sulfate Polyacrylamide Gel Electrophoresis (SDS-PAGE)

SDS-PAGE was carried out using 4% stacking gel and 12% separating gel according to the method described by Chen et al., Food Hydrocolloids, 2022, 128, 107547, which is incorporated herein by reference for its teaching regarding the same. Heated WP solution (85° C., 20 min) was diluted to a protein concentration of 2 mg/mL. The diluent was be mixed with the same volume of 2× Laemmli sample buffer containing 5% 2-Mercaptoethanol, and heated at 100° C. for 5 min. An aliquot (10 μL) of the mixture and protein standards (10-250 kDa, Precision Plus Protein™) was loaded into gels. Electrophoresis was run at 100 V for 2 hours. Afterwards, the gels were fixed (methanol:acetic acid:H₂O, 5:1:4), washed with water, stained with Bio-safe Coomassie Blue G-250 for 2 hours, distained with diluted acetic acid for 2 hours, and imaged.

### Size of Aggregates in Heated WP Solution

Heated WP solution (85° C. for 20 min) was carefully transferred to Norell NMR tubes (5 mm outer diameter, 4.2 mm inner diameter), and irradiated with synchrotron X-ray with 21 keV energy and 5×1012 mm⁻² s⁻¹ photo flux in Argonne National Laboratory (Lemont, IL). A 90 s measurement was used for USAXS, and 20 s was used for SAXS. The scattering from the solvent background and the sample holder was subtracted before analysis.

A multi-level unified fit (Greg Beaucage Unified fit citation) was used to fit the scattering data generated by the Irena package (Ilavsky and Jemian, Journal of Applied Crystallography, 2009, 42 (2), 347-353). This model assumes the particles (scatters) in the system have sizes of different dimensions (structural levels) and shapes with assumption that the interactions among scatters have limited effects on the scattering signals. The radius of gyration (R_{g}) and the structurally limited power law (P) of aggregates of various sizes can be acquired from the fitting method according to the following equation:

$$I ( Q ) = {\sum }_{i = 1}^{n} {G}_{i} exp ( \frac{- {q}^{2} {R}_{gi}^{2}}{3} ) + {{B}_{i} exp ( \frac{- {q}^{2} {R}_{g ( i - 1 )}^{2}}{3} ) [ \frac{{( erf ( \frac{{qR}_{gi}}{\sqrt{6}} ) )}^{3}}{q} ]}^{{P}_{i}} + {I}_{bkg} \qquad ( 1 )$$

Where i represents structural level, R_{g} is the intensity weighted average radius of gyration of scatters, Pᵢ is the power law exponent, Gᵢ is the Guinier scale, and Bᵢ is the perfector of power-law scattering at structural level i.

A modelling tool can also be used to analyse the size distribution of the scatters during gelation using Irena package with a limited number of bins in radii according to the following equation that considers the interference between scatters.

$$I ( Q ) = {\sum }_{k} {\vert Δρ \vert}^{2} {S}_{k} ( Q ) {\sum }_{{j}_{k}} {\vert {F}_{k} ( Q , {r}_{jk} ) \vert}^{2} {V}_{k} ( {r}_{jk} ) {f}_{k} ( {r}_{{j}_{k}} ) Δ {r}_{{j}_{k}} \qquad ( 2 )$$

where subscript j includes all bins in the size distribution and Δrⱼ is the width of bin j. Subscript k denotes different populations with each has a binding index jₖ. r is the radius of the scatter. |Δρ|² is the scattering contrast, S (Q) is the structure factor. F(Q,r) is the scattering form factor, V(r) is the volume of the scattering particle. f(r) is the volume size distribution calculated as:

$$f ( r ) = V ( r ) * N ( r ) = V ( r ) * {N}_{T} ψ ( r ) \qquad ( 3 )$$

V(r) is the volume of the scatter, N(r) is the number distribution, N_{T} is the total number of the scatter, and Y(r) is the probability of occurrence of scatter at size of r.

### Solubility of the WP Aggregates

Heated WP solution (85° C., 20 min) centrifuged (10,000×g, 15 min) to collect the supernatant, diluted with water followed by measurement of protein content using Bradford method (Kruger, The Protein Protocols Handbook, 17-24, 2009). Bovine serum albumin (BSA) was used as standard to build the calibration curve.

### Zeta Potential

WP solution with various degrees of deamidation before and after heating at 85° C. for 20 min diluted with water to a protein content of 0.15% (w/v) (Schneider et al. Food and Function, 2016, 7 (3), 1306-1318), loaded into folded capillary cell and analyzed with a Malvern zeta-sizer.

### Surface Hydrophobicity and Intrinsic Fluorescence

WP solution with various degrees of deamidation before and after heating at 85° C. for 20 min diluted to different concentrations (0.001 to 0.02%, w/v) using phosphate buffer. They were reacted with 2 mM ANS in the dark for 30 minutes before the measurement of fluorescence at 480 nm emission wavelength and 390 nm excitation wavelength using a FLS980 fluorometer (Edinburgh Instruments). The surface hydrophobicity (H₀) expressed as the slope of the curve reflecting the protein concentration-fluorescence intensity. For the intrinsic fluorescence, WP solutions were diluted to 0.01% with water followed by recording the intrinsic emission spectra in the range of 300-500 nm at a 294 nm excitation wavelength. The slit width was set at 5 nm for both excitation and emission (Heyn et al. European Polymer Journal, 2019, 120, 109211).

### Statistical Analysis

All experiments conducted in triplicates, the average data was shown. Statistical analysis conducted by one-way analysis of variance (ANOVA) using Turkey's comparison tests in Origin software (Northampton, Massachusetts, USA). The significant level of the data among different samples can be set at P<0.05.

### Results:

### Turbidity

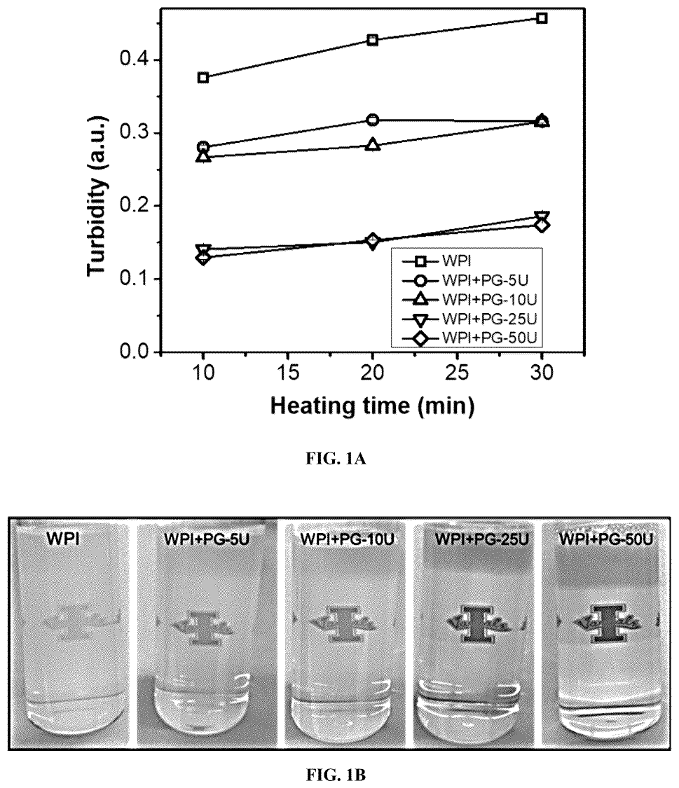

The turbidity of WP (5%, w/v) was increased with the increase in heating time (FIG. 1A and FIG. 1B). With 20 minutes heating at 85° C., the background letters became hardly recognizable. The addition of PG-500 at the level of 5 U/g and 10 U/g protein significantly reduced turbidity, and less changes were observed among different heating durations compared to those of the PG-500 free sample. Further, an increase in the PG-500 level to 25 U/g and 50 U/g protein resulted in a high-clarity solution. The turbidity was 0.13-0.18, which was ˜30-40% of PG-500 free sample. This demonstrates the efficiency of PG-500 in enhancing the heat stability of whey protein and reducing the turbidity of its solution at 5% concentration. The dose-dependent effect of PG-500 on the improvement of the clarity of whey protein solution was mainly due to a distinct degree of deamidation (FIG. 4a). There was no hydrolysis of whey protein observed as evidenced from a near zero DH value and the intact beta-lactoglobulin and alpha-lactalbumin bands in the SDS-PAGE (FIGS. 4b-4c).

### X-Ray Scattering

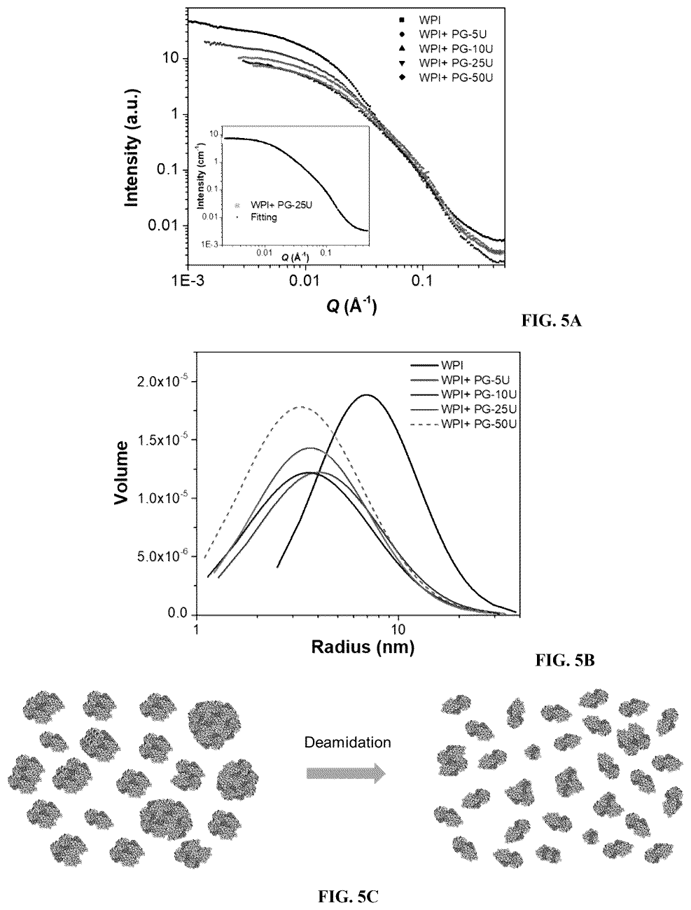

The size of the aggregates formed at 85° C. for 20 minutes was measured by using synchrotron x-ray scattering. The technique enables the characterization of protein aggregates or clusters in a relatively concentrated system compared to those of light scattering (Chen et al., Food Hydrocolloids, 2022, 26, 107449). At the region 0.001 Å-1<Q<0.03 Å-1 (FIG. 5a), the scattering intensity was weakened with the increased concentration of PG-500, indicating a reduced proportion of large aggregates in the system. The radius of aggregates in a sample with no PG-500 ranged between 2.5 nm and 35 nm, with a median value of ˜8 nm. Deamidation shifted the distribution to smaller values (2-10 nm range) with a median value of 4 nm. (FIG. 5b). The shift was more obvious in the samples when a higher amount of PG-500 was present. This indicates fewer protein molecules were included in the formed aggregates of PG-500 deamidated whey protein solution compared to those without the enzyme (FIG. 5c).

### Preparation of Whey Protein Beverage

Deamidated and non-deamidated WP solutions (5%) were added to water containing 7.5% sugar and stirred for 2 hours. The pH of the solution was adjusted to pH 7 using sodium bicarbonate followed by heating at 88° C. for 2 minutes, simulating a hot-fill process in the beverage industry. The solutions were stored at 5° C. The color of the beverages was measured on days 1, 7, 14, 21, 30, and 60 by taking four different random places and recorded as L*, a* and b*. The turbidity and protein solubility for each storage duration were also measured as described.

### Solubility

The solubility of the WP aggregates was increased from ˜32% in PG-500 free whey protein solution to ˜60% in those deamidated by PG-500 (FIG. 6a). No significant difference was observed among the samples containing different concentrations of PG-500. The increased solubility was most likely due to the decreased size of the aggregates and/or increased surface charge. Zeta-potential measurement found the surface charge of whey protein aggregates was increased from −31 mV to −29 mV after deamidation (FIG. 6b), indicating a lower surface charge, which was opposite to the expectation. However, PG-500 deamidated whey proteins had a higher surface charge before heating than the whey protein alone sample. This would provide stronger electrostatic repulsion among whey proteins during heating and contribute to the formation of smaller aggregates with higher stability. In addition to electrostatic interactions, hydrophobic interaction also plays a significant role in protein aggregation. The surface hydrophobicity of the WP aggregates decreased gradually with the increases of PG-500 level (FIG. 6c), probably due to the limited denaturation of whey proteins or increased deprotonation of amino acid side chains. Intrinsic fluorescence spectroscopy of the aggregates showed a decreased fluorescence intensity with the increase of PG-500 level (FIG. 6d). This indicates a more hydrophilic environment of microdomain surrounding tryptophan residues when whey protein was deamidated (Chen and Campanella, Food Hydrocolloids, 2022, 128, 107547), and agrees well with the surface hydrophobicity data. Heating may dilute the changes in whey proteins induced by deamidation. Thus, the surface hydrophobicity and intrinsic fluorescence of whey protein samples before heating were also measured. The surface hydrophobicity was increased slightly, whereas the intensity of intrinsic fluorescence was decreased together with the red shift of the wavelength at maximum intensity after deamidation (FIGS. 6c-6d). These imply whey proteins were partially unfolded after deamidation.

### Storage Stability

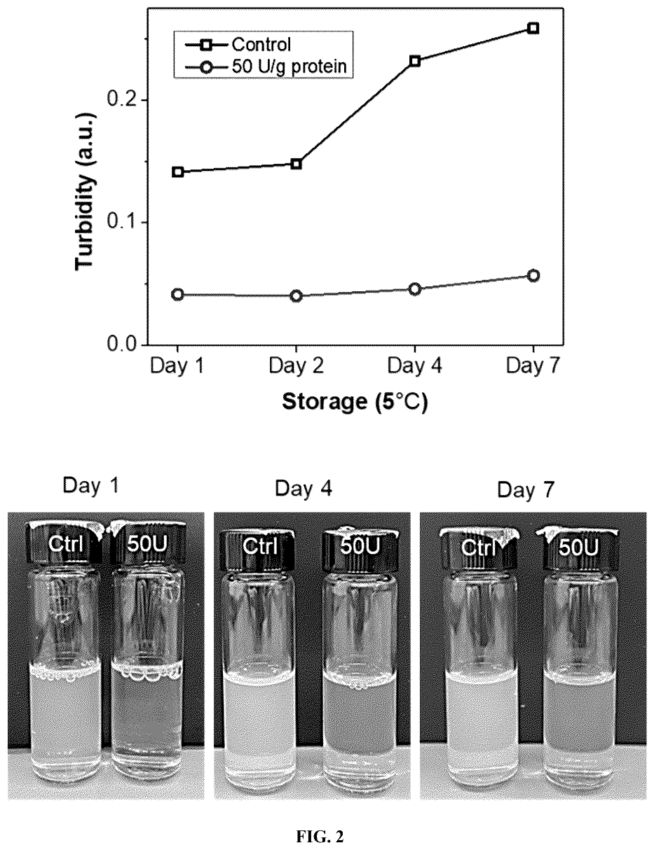

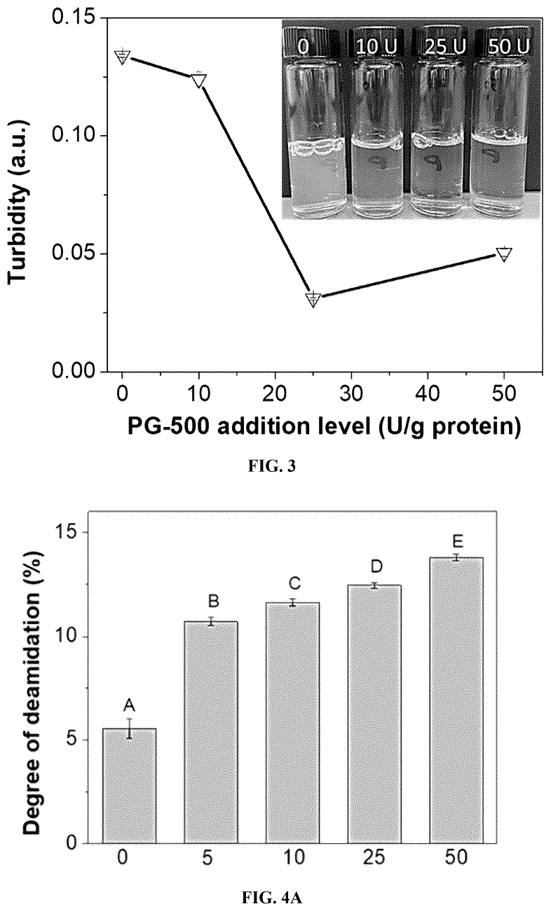

The heated WP solution (85° C., 20 minutes) with and without PG-500 treatment were stored at 5° C. for up to 1 week, and longer storage time may cause spoilage of the sample. The changes in turbidity were measured during the storage period and shown as FIG. 2. With storage time proceeds, the turbidity of the WPI-control sample and PG-500 treated samples increased, indicating the formation of large particles. Compared to the control samples, the PG-500 treated sample had much less turbidity throughout the entire storage period. After one week, the turbidity of the PG-500 treated sample was 0.056, whereas the control sample was 0.26. This clearly indicates that PG-500 treated samples are able to maintain high clarity after storage at refrigeration conditions.

### Pg-500 Treated Whey Protein Powder for Direct Application

For commercial applications, ingredients in powder form are convenient to use, transport, and store. The PG-500 treated whey proteins were freeze-dried and reconstituted in water prior to heating at 85° C. for 20 minutes. With the increase in the amount of PG-500, the turbidity of the whey protein solution was first decreased and then increased. At 25-50 U/g PG-500, the turbidity was only 22-40% of the control sample. The whey proteins can be derived from sweet whey protein.

The following data clearly demonstrates the potential of developing a PG-500 treated whey protein powder for high-clarity beverage applications.

### Thermal Stability

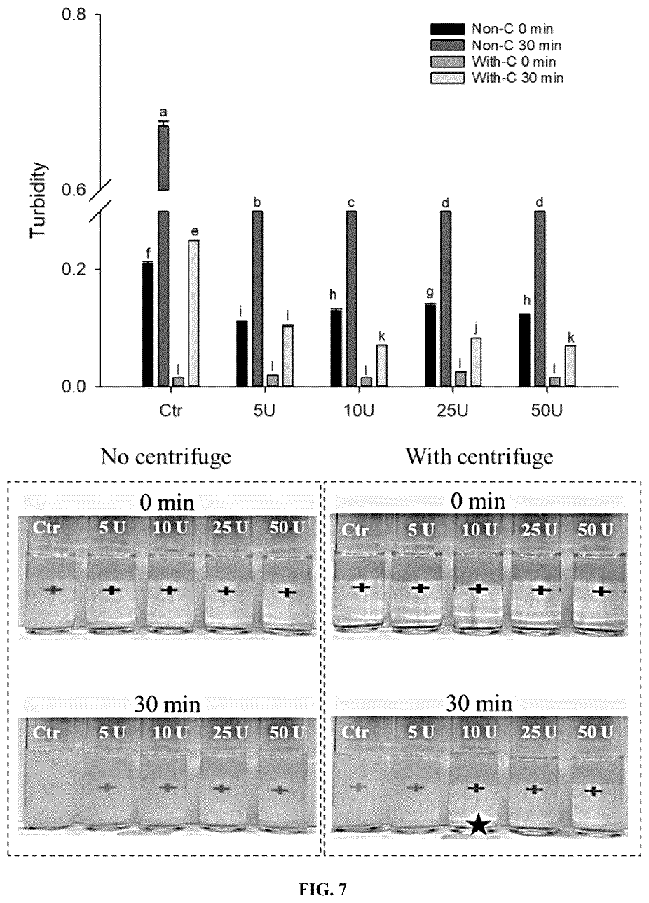

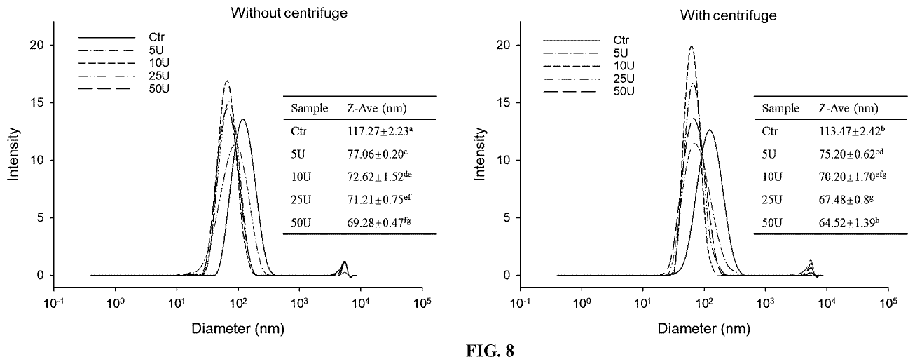

After deamidation of 5% WP solution by 5-50 U/g PG-500, the solutions were spray dried to obtain the whey protein powder prior to thermal stability testing. After re-dissolving the powder to water and making 5% solution, the turbidity was significantly less in deamidated whey proteins compared to the non-deamidated counterpart (FIG. 7, top). Further heating the samples at 75° C. for 30 minutes increased the turbidity of all samples, and deamidated samples showed less turbidity. Considering some proteins were denatured and formed large aggregates during spray drying, the 5% re-suspended WP solutions were centrifuged to remove large aggregates, followed by heating at 75° C. for 30 minutes (FIG. 11). The deamidated whey proteins were less turbid compared to the control, and 10-25 U/g samples showed the least turbidity. These findings demonstrate that the deamidated whey proteins retained thermal stability after spray drying. Particle size measurement showed, either with or without centrifugation, that the size of the aggregates was decreased with the increase of PG-500. For instance, the average size was 117.3 nm in the control samples, which decreased to ˜70 nm in the 50 U-treated samples (FIG. 8, left). Centrifugation hardly changed the particle size, which indicates small proportions of large aggregates were formed during the spray drying process (FIG. 8, right).

### Differential Scanning Calorimetry (DSC) for Thermal Stability

To characterize the effects of spray drying and deamidation on the thermal stability of whey protein, DSC was utilized to assess the relevant thermodynamic parameters, as summarized in Table 1. Enthalpy serves as an indicator of the proportion of ordered and stable protein structures, while peak temperature reflects the thermal stability of proteins. The results demonstrated that spray drying resulted in a lower denaturation enthalpy of 0.35 J/g, indicating that less energy was required for denaturation in this state. Following spray drying, deamidation reduced the onset temperature of the denaturation peak by 1.11-1.44° C. and increased the peak temperature by 0.62-1.18° C., with these trends becoming more pronounced as the concentration of PG-500 increased. This indicated that thermal denaturation began at lower temperatures due to the disruption of the whey protein's native state, whereas more substantial structural changes were observed at elevated temperatures. Thus, enzymatic deamidation exhibited a protective effect on whey protein powder against thermal degradation, with increasing enzyme concentrations amplifying this protective role.

Table 1 illustrates the onset temperature, peak temperature, and enthalpy of whey protein powder after spray drying. The whey proteins were deamidated with different amounts of PG-500 prior to spray drying. Different letters in a and d denote significant differences among the data (P<0.05).

TABLE 1

| | Onset | Peak | |
|---|---|---|---|
| | Temperature | Temperature | Enthalpy |
| | (° C.) | (° C.) | (J/g) |
| Ctr-without heat | 70.06 ± 0.04^{cd} | 73.39 ± 0.04^{d} | 0.71 ± 0.01^{a} |
| Ctr | 71.91 ± 0.42^{a} | 76.46 ± 0.26^{c} | 0.35 ± 0.02^{b} |
| 5 U | 70.80 ± 0.10^{b} | 77.08 ± 0.05^{b} | 0.27 ± 0.01^{c} |
| 10 U | 70.55 ± 0.13^{bc} | 77.29 ± 0.07^{b} | 0.26 ± 0.01^{cd} |
| 25 U | 70.48 ± 0.20^{bc} | 77.25 ± 0.09^{b} | 0.26 ± 0.01^{cd} |
| 50 U | 70.47 ± 0.26^{bc} | 77.64 ± 0.16^{a} | 0.23 ± 0.01^{d} |

^{a, b, c} and ^{d}different letters denote the significant differences among the data in the same column (P < 0.05).

### Transmission Electron Microscopy (TEM) (Morphology)

To investigate the structure, morphology, and size of WP aggregates following spray drying, TEM was employed on both native and deamidated WP samples after thermal treatment. FIG. 9 presents the TEM image of WP at a magnification of ×80.0 k. Compared with the obvious severe aggregation of PG-500 free WP, the aggregate size of the deamidated WP particles showed a decreasing trend with the increase of PG-500 dosage. Deamidation also improved the light transmittance and clarity of WP particles, and more evenly distributed small protein aggregates appeared, which manifested as improved thermal stability and improved clarity of the WP solution.

### Zeta Potential (Solution Stability)

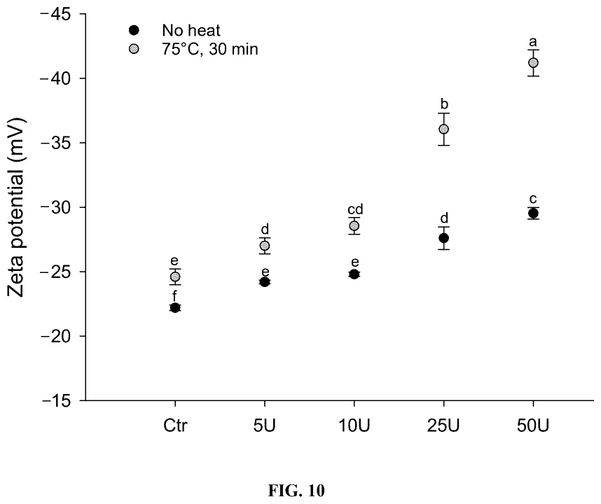

To evaluate surface charge levels, the zeta potential of WP aggregates was measured (FIG. 10). The addition of PG-500 showed a positive correlation with an increase in the absolute value of the zeta potential in both non-heated and heated WP solutions. After 75° C. and 30 minutes of heating, the zeta potential absolute value significantly increased from ˜25 mV in the free PG-500 to ˜41% in the 50 U/g PG-500-deamidated whey protein solution. These changes in zeta potential suggested enhanced electrostatic repulsion during heating, facilitating the formation of smaller aggregates and improving solution stability.

While specific embodiments of the subject invention have been disclosed herein, the above specification is illustrative and not restrictive. While this invention has been particularly shown and described with references to preferred embodiments thereof, it will be understood by those skilled in the art that various changes in form and details may be made therein without departing from the scope of the invention encompassed by the appended claims. Many variations of the invention will become apparent to those of skilled art upon review of this specification. Unless otherwise indicated, all numbers expressing reaction conditions, quantities of ingredients, and so forth, as used in this specification and the claims, are to be understood as being modified in all instances by the term “about.” Accordingly, unless indicated to the contrary, the numerical parameters set forth herein are approximations that can vary depending upon the desired properties sought to be obtained by the present disclosure.

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range.

The term “substantially” can allow for a degree of variability in a value or range, for example, within 90%, within 95%, or within 99% of a stated value or of a stated limit of a range.

The terms “a,” “an,” or “the” are used to include one or more than one unless the context clearly dictates otherwise. The term “or” is used to refer to a nonexclusive “or” unless otherwise indicated. In addition, the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. The terms “including” and “having” are defined as comprising (i.e., open language).

Those skilled in the art will recognize that numerous modifications can be made to the specific implementations described above. The implementations should not be limited to the particular limitations described. Other implementations may be possible.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

What is claimed is:

1. A method for producing a deamidated sweet whey protein powder (DSWPP), which method comprises:
(i) heating an aqueous solution of a concentrated sweet whey and an enzyme protein glutaminase (e.g., PG-500) at about 40° C. to about 60° C. to obtain a solution of deamidated sweet whey; and
(ii) either spray-drying or freeze-drying the solution of deamidated sweet whey,
whereupon the deamidated sweet whey protein powder (DSWPP) is produced.

2. The method of claim 1, wherein the concentrated sweet whey comprises a protein content of about 5% w/v to about 15% w/v.

3. The method of claim 1, wherein an amount of the protein glutaminase used is from about 5 U/g protein to about 50 U/g protein.

4. The method of claim 1, wherein the DSWPP comprises about 70% to about 90% of proteins.

5. The method of claim 1, wherein the solution of step (i) is heated for about 1 hour.

6. A whey protein beverage comprising about 3% by weight to about 10% by weight of a whey protein, wherein the whey protein is from a deamidated sweet whey protein powder (DSWPP), a sweetener and having a turbidity value of less than about 0.1 a.u.

7. The whey protein beverage of claim 6, wherein the sweetener is a sugar or an artificial sweetener.

8. The whey protein beverage of claim 6, wherein the whey protein beverage further comprises one or more additives.

9. The whey protein beverage of claim 8, wherein the one or more additives selected from a nutrient, a herbal supplement, a flavoring agent, a coloring agent, and a combination of two or more thereof.

10. The whey protein beverage of claim 6, wherein the whey protein beverage has a turbidity value of less than about 0.1 a.u. for a time period from about a week to about 1.5 months during storage with or without refrigeration.

11. The whey protein beverage of claim 6, wherein the DSWPP is prepared in accordance with the method of claim 1.

12. The whey protein beverage of claim 6, wherein the whey protein beverage can be a protein shake or a protein cream.

13. A protein shake comprising the whey protein beverage of claim 6.

14. A protein cream comprising the whey protein beverage of claim 6.

15. A method of producing a whey protein beverage, which method comprises:
a. preparing an aqueous solution of a deamidated sweet whey protein powder (DSWPP) to achieve a protein content of about 3% w/v to about 10% w/v in the solution;
b. combining the aqueous solution of DSWPP with a sweetener to produce a whey protein beverage; and
c. subjecting the whey protein beverage to an ultra-high temperature (UHT) processing.

16. The method of claim 15, wherein the method further comprises an addition of one or more additives.

17. The method of claim 15, wherein the whey protein beverage is produced at a neutral pH.

18. The method of claim 15, wherein the whey protein beverage has a turbidity of less than about 0.1 a.u.

19. The method of claim 15, wherein the DSWPP is prepared in accordance with the method of claim 1.

20. A method for producing a deamidated whey protein powder (DWPP), which method comprises:
(i) heating an aqueous solution of a whey protein comprising a protein content of about 5% w/v to about 15% w/v and an enzyme protein glutaminase (e.g., PG-500) at about 40° C. to about 60° C. to obtain a solution of deamidated whey protein; and
(ii) either spray-drying or freeze-drying the solution of deamidated whey protein,
whereupon the deamidated whey protein powder (DWPP) is produced.

21. The method of claim 20, wherein an amount of the protein glutaminase used is from about 5 U/g protein to about 50 U/g protein.
